(19) United States
(12) Patent Application Publication
(10) Pub. No.: US 2022/0061641 A1
(43) Pub. Date: Mar. 3, 2022

(54) APPARATUS FOR THERMALLY STABLE BALLOON EXPANSION

(71) Applicant: CAPSOVISION, Inc, Saratoga, CA (US)

(72) Inventors: Mikael TROLLSAS, San Jose, CA (US); Phat TRINH, San Jose, CA (US); Amy FREITAS, Morgan Hill, CA (US)

(21) Appl. No.: 17/292,429

(22) PCT Filed: Oct. 11, 2018
(86) PCT No.: PCT/US18/55396

§ 371 Date: May 7, 2021

(51) Int. Cl. A61B 1/04; A61B 1/00; C11D 3/10

(52) U.S. Cl. CPC A61B 1/041; C11D 3/10; A61B 1/00082

(57) ABSTRACT

The present invention discloses a capsule device with specific gravity control. The capsule device comprises a capsule unit adapted to be ingested by a human subject, and an inflatable balloon comprising an effervescent formulation inside the inflatable balloon. The effervescent formulation comprises sodium carbonate, potassium bicarbonate or both with excess citric acid and the inflatable balloon is attached to the capsule unit. After the capsule unit with the inflatable balloon attached is swallowed, the inflatable balloon starts to inflate so as to lower the specific gravity, of the combined device comprising a combination of the capsule unit and the inflatable balloon, when the inflatable balloon is exposed to body liquid and the body liquid gets in touch with the effervescent formulation inside the inflatable balloon.

### CROSS REFERENCE TO RELATED APPLICATIONS

The present invention is related to PCT Patent Application, Serial No. PCT/US13/66011, filed on Oct. 22, 2013, PCT Patent Application, Serial No. PCT/US14/68601, filed on Dec. 4, 2014 and U.S. patent application Ser. No. 14/659,832, filed on Mar. 17, 2015. The PCT Patent Applications and U.S. patent application are hereby incorporated by reference in their entireties.

### FIELD OF THE INVENTION

The present invention relates to diagnostic imaging inside the human body or any other living creature. In particular, the present invention relates to an in-vivo capsule that uses effervescent formulations for a balloon coated to cause thermally stable balloon expansion and to achieve desired specific gravity control of the capsule via balloon inflation and deflation.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS sensor array, and transfer the image data as an electrical signal to the proximal end through a cable. Endoscopes allow a physician or a veterinary physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot easily reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia.

An alternative in vivo image sensor that addresses many of these problems is the capsule endoscope. A camera is housed in an ingestible capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage. The capsule device 110 includes illuminating system 12 and a camera that includes optical system 14 and image sensor 16. A semiconductor nonvolatile archival memory 20 may be provided to allow the images to be stored and later retrieved at a docking station outside the body, after the capsule is recovered. Capsule device 110 includes battery power supply 24 and an output port 26. Capsule device 110 may be propelled through the gastrointestinal (GI) tract by peristalsis.

Illuminating system 12 may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14, which may include multiple refractive, diffractive, or reflective lens elements, provides an image of the lumen walls (100) on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (A/D) converter, which may be provided inside the sensor (as in the current case), or in another portion inside capsule housing 10. The A/D unit may be provided between image sensor 16 and the rest of the system. LEDs in illuminating system 12 are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval. While FIG. 1 illustrates a capsule endoscope with an archival memory to store captured images, the capsule endoscope may also be equipped with a wireless transmitter to transmit the captures to an external receiver.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine the images and correctly identify any anomaly.

When the capsule device travels through the GI tract, the capsule device will encounter different environments. It is desirable to manage the capsule device to travel at a speed that sufficient sensor data (e.g., images) can be collected at all locations along the portions of the GI tract which are of interest, without wasting battery power and/or data storage by collecting excessive data in some locations. In order to manage the capsule device to travel at a relatively steady speed, techniques have been developed to change the capsule specific gravity during the course of travelling through the GI tract. In some environments, it is desirable to have a capsule with higher specific gravity. In other environments, it may be desirable to have a capsule with lower specific gravity. For example, it is desirable to configure the capsule device to have a lower specific gravity when the capsule device travels through the ascending colon. On the other hand, it may be desirable to configure the capsule device to have a higher specific gravity when the capsule device travels through the stomach or the descending colon, in particular if those anatomies are filled with liquid. However, techniques based on specific gravity or density control may not work reliably due to various reasons. For example, the change of specific gravity or density may not have to take place at the intended section of the GI tract. Therefore, the location of the capsule device inside the GI tract has to be monitored or estimated. However, the location of the capsule device usually cannot be accurately determined without the use of additional equipment outside the patient's body. Therefore, it is desirable to develop reliable means to manage the capsule device to travel at a relatively steady speed in the GI tract.

### BRIEF SUMMARY OF THE INVENTION

A capsule device with heat-stable specific gravity control is disclosed. The capsule device comprises a capsule unit adapted to be swallowable by a human subject and an inflatable balloon comprising a heat-stable effervescent formulation inside the inflatable balloon. The heat-stable effervescent formulation is substantially free from thermal degradation up to a transportation temperature or shelf-life temperature belonging to a temperature range including 40° C., and wherein the inflatable balloon is attached to the capsule unit. After the capsule unit with the inflatable balloon attached is swallowed, the inflatable balloon starts to inflate so as to lower specific gravity of a combination of the capsule unit and the inflatable balloon when the inflatable balloon is exposed to body fluid and the body fluid gets in touch with the heat-stable effervescent formulation inside the inflatable balloon.

The heat-stable effervescent formulation may comprise sodium carbonate, potassium bicarbonate or both with an excess acid. The excess acid selected can be crystalline/semi-crystalline, anhydrous, low molecular weight, and water soluble. For example, the excess acid may belong to a group comprising citric acid, tartaric acids and monocalciumphosphate (Ca(H₂PO₄)₂).

For the excess acid, any excess acid relative to a base corresponds to at least two times a balanced stoichiometric molar ratio can be used. When the sodium carbonate is used, the sodium carbonate can be contained in the inflatable balloon with the excess acid relative to a base of at least about five times a balanced stoichiometric molar ratio. When 10-25 mg potassium bicarbonate is used, the 10-25 mg potassium bicarbonate can be contained in the inflatable balloon with an excess acid relative to a base of at least about five times a balanced stoichiometric molar ratio.

In one embodiment, the inflatable balloon is enclosed in an enteric or enteric coated shell to control inflation starting time. When the potassium bicarbonate is used, the excess acid selected is crystalline or semi-crystalline, anhydrous, low molecular weight, and water soluble. For example, the excess acid can be selected from a group comprising citric acid, tartaric acids and monocalciumphosphate (Ca(H₂PO₄)₂) mixed with polyethylene glycols (PEG) or an alternative desiccant to further control the inflation starting time. For example, about 10-25 mg potassium bicarbonate along with the excess acid relative to a base of about three six times a balanced stoichiometric molar ratio and mixed with about 5-25 mg PEG or alternative desiccant are contained in the inflatable balloon. The enteric or enteric coated shell may correspond to a half shell or a full shell to enclose the inflatable balloon between the half shell and a portion of the capsule unit.

In one embodiment, when the potassium bicarbonate is used, the potassium bicarbonate along with the excess acid is mixed with polyethylene glycols (PEG) to further control the inflation starting time. In one embodiment, the inflatable balloon comprises a thickness of about 0.5-5 mil. The inflatable balloon comprises a thickness about 0.5-2 mil. In one embodiment, the capsule unit comprises a camera to capture images while the capsule unit travels in a gastrointestinal tract of the human subject.

In one embodiment, the capsule device is stored and/or transported through including a temperature range from 40° C. to 60° C. For example, the temperature range corresponds to 40° C. to 60° C. In another embodiment, the capsule device is stored and/or transported through includes a temperature range from 40° C. to 50° C.

In another aspect of the present invention is directed to the heat-stable effervescent formulation, which comprises a target effervescent with an excess acid. The excess acid selected is crystalline/semi-crystalline, anhydrous, low molecular weight, and water soluble. Furthermore, the heat-stable effervescent formulation is substantially free from thermal degradation in an environment below a transportation temperature or shelf-life temperature belonging to a temperature range including 40° C. The target effervescent may comprise sodium carbonate, and/or potassium bicarbonate. Any excess acid relative to a base corresponds to at least two times a balanced stoichiometric molar ratio. The excess acid belongs to a group comprising citric acid, tartaric acids and monocalciumphosphate (Ca(H₂PO₄)₂).

### BRIEF DESCRIPTION OF THE DRAWINGS

FIG. 1 shows schematically a capsule camera system in the GI tract, where archival memory is used to store captured images to be analyzed and/or examined.

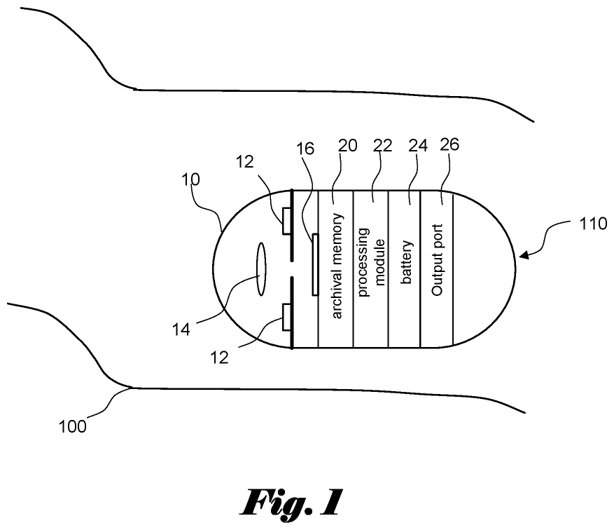

FIG. 2 illustrates the comparison of thermal stability among sodium bicarbonate, sodium carbonate and potassium bicarbonate at 50° C. as a function of time.

FIG. 3 illustrates an example of CO₂ (g) inflation curve using sodium carbonate as the effervescent base and citric acid with a balanced stoichiometric ratio.

FIG. 4 illustrates an example of CO₂ (g) inflation curve using sodium carbonate as the effervescent base with a stoichiometric excess of citric acid.

FIG. 5 illustrates the comparison of CO₂ (g) inflation curve for sodium carbonate and potassium bicarbonate, both with a stoichiometric excess of citric acid.

FIG. 6 illustrates the CO₂ (g) inflation curve for potassium bicarbonate with excess citric acid, where an enteric coated shell is used to control the balloon inflation initiation.

FIG. 7 illustrates the CO₂ (g) inflation curve for potassium bicarbonate plus polyethylene glycols (PEG) with excess citric acid, where both the PEG and an enteric coated shell are used to control the balloon inflation initiation.

FIG. 8 illustrates the CO₂ (g) inflation curve for sodium carbonate with excess citric acid, where an enteric coated shell is used to control the balloon inflation initiation.

FIG. 9 illustrates the CO₂ (g) inflation curve for a thin balloon containing potassium bicarbonate and excess citric acid with different quantities of polyethylene glycol (PEG), where both the PEG and an enteric coated shell are used to control the balloon inflation initiation.

FIG. 10 illustrates the CO₂ (g) gas inflation curve for a thin balloon and regular balloon containing potassium bicarbonate with excess citric acid plus polyethylene glycol (PEG), where both the PEG and an enteric coated shell are used to control the balloon inflation initiation. In addition, the balloon thickness helps control the balloon deflation timing.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention.

Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention.

The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

In U.S. Pat. Nos. 7,192,397 and 8,444,554, a capsule device with specific gravity about 1 is disclosed. When the capsule device has a specific gravity about 1, the device will suspend or float in the liquid in the gastrointestinal (GI) track such as in the stomach or in the colon. As disclosed in U.S. Pat. Nos. 7,192,397 and 8,444,554, the capsule device will be carried through the body lumen by a flow of liquid through the body lumen when the capsule device has a specific gravity about 1. However, for an in vivo capsule device, after the capsule device is swallowed by a patient, the capsule device first goes through the pharynx and esophagus into the stomach and the stomach may be filled with liquid. If the specific gravity of the capsule device is less than 1 or the capsule device has a lighter density than the liquid, it will float on the surface of the liquid inside stomach. Thus, it is not conducive for the capsule device to transit through the pylorus into the small bowel. Therefore, it is desirable to cause the specific gravity of the capsule endoscope greater than one when the capsule endoscope is in the stomach.

For a capsule device with an image sensor, it is critical to have a steady and consistent travelling velocity inside different regions of the GI tract, e.g. stomach, small bowel, ascending and descending colons so that smooth and stable images and video can be obtained. The travelling velocity of the capsule camera depends on many factors including but not limited to regional gastrointestinal motility and preparation, anatomical position, physical activity, hydration, gravitational force, buoyancy and viscous drag of the surrounding fluids. After the capsule device is swallowed, it is propelled into the esophagus. Gravitation and peristaltic waves in the esophagus move the camera into the stomach. After the capsule device passes the cardia and enters the stomach with fluid or limited amount of fluids, the balance among gravitational force, anatomical position, buoyancy and drag from fluids starts to affect its travelling velocity and transit time. The migrating myoelectric cycle (MMC) can be divided into four phases. Generally phase 1 lasts between 30 and 60 minutes with rare contractions while phase 2 lasts between 20 and 40 minutes with intermittent contraction. Phase 3, or the housekeeping phase, typically lasts between 10 and 20 minutes with intense and regular contractions for short period. The housekeeping wave sweeps most of the undigested material out of the stomach and into the small bowel. The last phase, phase 4, typically lasts between 0 and 5 minutes and occurs between phase 3 and phase 1 of two consecutive cycles. For the capsule device to travel aborally at a desired rate it is desirable that the capsule have a specific gravity greater than 1 (e.g., 1.1) to help overcome buoyance and drag from the surrounding fluids. If phase 3 is detected through image motion detection or accelerometer, the specific gravity can be pushed to a value less than one (e.g., 0.97) for the capsule device to float to the top and to retake the video in a more stable phases.

In the small intestine, BER (basic electrical rhythm) is around 12 cycles per minute in the proximal jejunum and decreases to around 8 cycles per minutes in the distal ileum. There are three types of smooth muscle contractions: peristaltic waves, segmentation contractions and tonic contractions. Normally, peristalsis will propel the capsule device towards large intestines. Since the small intestine twists and turns around between the stomach and the large intestine, the capsule device may sometimes be trapped at corners and turns. In this case, motion detection may be used to detect such situation. Accordingly, density-changing mechanisms can be used to slightly change the balance between gravity and buoyancy so that the capsule device can leave the trap sooner before the next peristalsis.

While the large intestine is one organ, it demonstrates regional differences. The proximal (ascending) colon serves as a reservoir and the distal (transverse and descending) colon mainly performs as a conduit. The character of the luminal contents impacts the transit time. Liquid passes through the ascending colon quickly, but remains within the transverse colon for longer periods of time. In contrast, a solid meal is retained by the cecum and ascending colon for longer periods than a liquid diet. In the ascending colon, retrograde movements are normal and occur frequently. In order for the buoyant force to overcome the gravitational force and retropulsion, the specific gravity of the capsule device according to an embodiment of the present invention is ideally decreased to less than less than one (e.g., 0.99 or less) before or after the capsule enters the ascending colon. Alternatively, the density of the capsule device as a whole has lighter density than the surrounding fluid. In the descending colon and rectum, propulsive contractions prevail. The capsule device is carried aborally towards the rectum by the natural propulsion. However, increasing the specific gravity of the apparatus to larger than one (e.g., 1.1 or larger) could shorten the transit time and allow a smooth and steady motion. Therefore, it is desirable to cause the specific gravity to be greater than one when the capsule endoscope reaches the descending colon.

In order to properly set the specific gravity or the density of the capsule device, ideally the capsule would know which regions of the GI tract it is located. There are various know region detection methods in the literature. The region detection methods include estimated transit time (e.g., less than about 1 hour in the stomach and about 2-4 hours in the small bowel), identification of image contents based on captured images by the capsule device, motion detection based on the captured images by the capsule device, pH detection (pH value increasing progressively from the stomach (1.5-3.5) and the small bowel (5.5-6.8) to the colon (6.4-7.4), pressure sensor (higher luminal pressure from peristaltic motion in the colon than that in the small bowel) and colonic microflora. The ascending colon has a larger diameter than other regions besides the stomach. The size may be detected by the methods disclosed in U.S. Patent Publications, Series No. 2007/0255098, published on Nov. 1, 2007, U.S. Patent Publications, Series No. 2008/0033247 published on Feb. 7, 2008 and U.S. Patent Publications, Series No. 2007/0249900, published on Oct. 25, 2007.

Accordingly, in PCT Patent Application, Serial No. PCT/US13/66011, a method is disclosed to configure the capsule device to have a specific gravity (SG) larger than 1 or a density higher than the liquid in the stomach when the capsule device is in the stomach. After the capsule passes through the small bowel and enters the cecum, it has to transit through the ascending colon. PCT Patent Application, Serial No. PCT/US13/66011 further discloses a method to cause the capsule device to have a specific gravity less than 1 or to have a lighter density than the liquid in the cecum and ascending colon.

In order to control the SG, PCT Patent Application, Serial No. PCT/US13/66011 discloses a capsule with an inflatable balloon, which is a deformable membrane, containing effervescent material. The inflatable balloon is expandable and made of material that is permeable to external water, such as intestinal fluids or prep medications. Furthermore, an enteric coating is applied to the outer surface of the inflatable balloon. The enteric coating may also cover the entire capsule system. Furthermore, instead of coating the balloon, the balloon may be put into a capsule shell, which will dissolve in the stomach or small bowel within about 30 minutes of swallowing, unless the capsule shell is enteric or coated with an enteric, in which case it will not dissolve in the low pH of the stomach, but disintegrate in the higher pH environment of the small bowel or colon. When the capsule device approaches the terminal ileum or the cecum, the enteric coating will disintegrate through swelling or dissolution due to the higher pH level. With the enteric coating disintegrated, intestinal fluids will gradually get into the deformable member. When the water of the fluid makes contact with the effervescent formulation, gas will be generated to expand the deformable member. While a small amount of fluid gets into the deformable member, the gas generated is able to expand the deformable member so that the capsule device as a whole has a specific gravity less than one.

The effervescent material should be in contact with the semipermeable membrane of the deformable member so that water that diffuses through the membrane will reach the effervescent material as designed. The effervescent material may be a powder or dispersion that coats a portion of the inside surface of the membrane or it might comprise granules that rest on the surface of the membrane.

For controlling the specific gravity of the capsule device, an inflatable device (e.g. a balloon containing effervescent materials) is often used. The inflatable balloon usually is attached to the capsule. An enteric coating is applied to the outer surface of the inflatable shell to delay the time to inflate until the capsule reaches or about to reach an intended anatomic location (e.g. after leaving the stomach). There are various effervescent materials being used in the inflatable balloon. For example, sodium bicarbonate has been used as one component in the effervescent mixture that generates CO₂ (g) to inflate the attached balloon in-situ. While sodium bicarbonate or sodium bicarbonate mixtures can produce satisfactory results when the material is handled in a thermally controlled environment (e.g. around 25° C.) during shipment and storage, sodium bicarbonate becomes thermally unstable and starts to degrade at around 40° C. and more rapidly at 50° C. Therefore, such effervescent material is not well suited for the warmer geographies or shipping in general. Accordingly, the present invention explores other alternatives that may be more stable in the warmer environments.

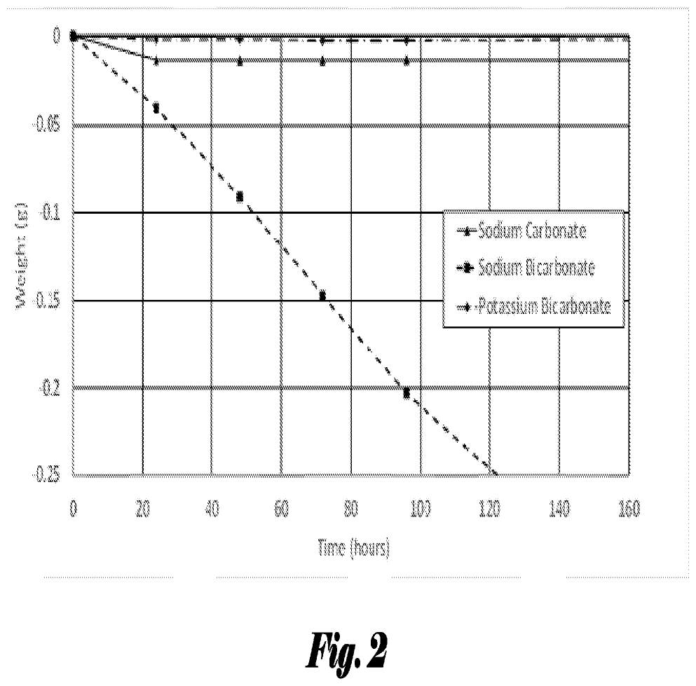

In order to identify suitable alternative for sustaining warmer geographies, thermal stability of sodium bicarbonate, sodium carbonate and potassium bicarbonate at 50° C. is compared as shown in FIG. 2. The horizontal axis represents the time (in hours) that the underlying material is subject to high temperature (i.e., 50° C.). The vertical axis corresponds to the weight loss (in gram) of the underlying material due to high temperature (i.e., 50° C.). Compared to sodium bicarbonate, sodium carbonate and potassium bicarbonate are much more stable in term of thermal degradation. In particular, there is almost no thermal degradation for potassium bicarbonate. Therefore, both sodium carbonate and potassium bicarbonate can be used as a candidate effervescent base to achieve thermal stability.

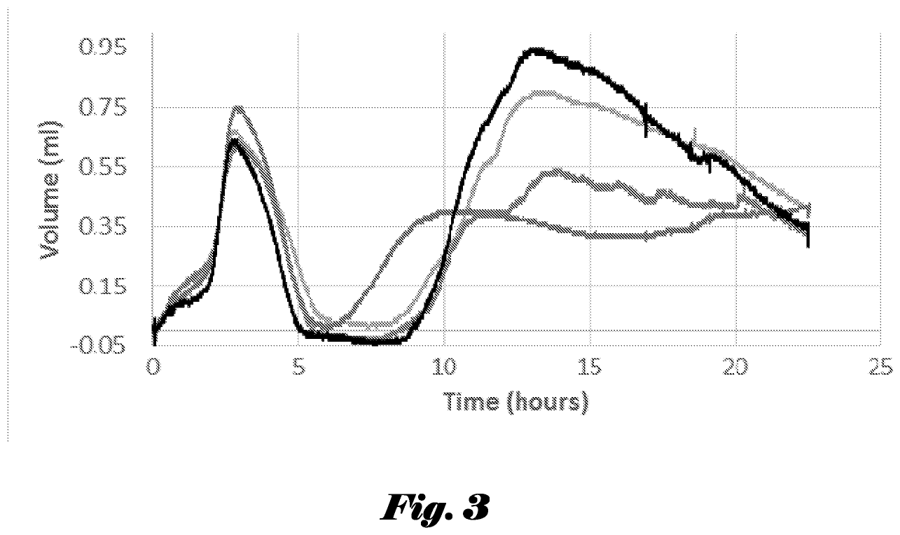

Accordingly, in one study, the sodium carbonate is used as a substitute for sodium bicarbonate so that it can be more stable in the hostile environment during shipment or storage. FIG. 3 illustrates an example of CO₂ (g) inflation/deflation curve using sodium carbonate as the effervescent base. This study uses a 2-mil PEBAX balloon containing sodium carbonate mixture having a total weight of about 26 mg. The sodium carbonate mixture consists of sodium carbonate and citric acid. Among the mixture, 12 mg (0.11 mol) are sodium carbonate (106 g/mol) and 14 mg (0.07 mol) is citric acid (192 g/mol) which gives an acid to base molar ratio of about 2:3 (0.07/0.11).

The molar ratio for this reaction requires 2 molecules of citric acid for every 3 molecules of sodium carbonate according to the balanced equation (1) below:

Balanced Equations:

2C₆H₈O₇+3Na₂CO₃→2Na₃C₆H₅O₇+3CO₂+3H₂O. (1)

C₆H₈O₇+3KHCO₃→K₃C₆H₅O₇+3CO₂+3H₂O. (2)

For the reaction in Equation (1) and (2) the rates of CO₂ (g) generation have the following dependencies on the concentrations of the two reactants:

rate_{c}=k[C₆H₈O₇]^{a}[Na₂CO₃]^{b}

rate_{f}=k[C₆H₈O₇]^{c}[KHCO₃]^{e},

which means that for a constant total volume of CO₂ (g) generated, an increase in the initial concentration of citric acid (C₆H₈O₇) would raise the rates of which CO₂ (g) is produced.

As shown in FIG. 3, the CO₂ (g) inflation/deflation curves using balanced sodium carbonate/citric acid mixtures result in an undesired bimodal balloon inflation behavior, where the CO₂ (g) volume rises again after an initial inflation-deflation cycle. A more desirable CO₂ (g) balloon inflation curve would have a single inflation (i.e., mono modal) and the CO₂ (g) volume would not rise again after the balloon has deflated. Therefore, while sodium carbonate as a base for effervescent mixture is more stable in terms of thermal degradation, the regular stoichiometrically balanced sodium carbonate mixture does not exhibit a desirable mono-modal CO₂ (g) balloon inflation behavior. Accordingly, the present invention further exploits possible effervescent materials/mixtures that may offer the desired thermal stability as well as satisfactory mono-modal gas inflation behavior.

In the present invention, effervescent mixtures with excess citric acid are disclosed as candidates for thermal stability as well as satisfactory mono-modal gas inflation behavior. In one embodiment, sodium carbonate with excess citric acid is used as the effervescent formulation. For example, sodium carbonate mixture with excess citric acid can be used, where the portion of citric acid is much larger than the balanced stoichiometric molar ratio of 2:3 (citric acid/sodium carbonate). In one embodiment, the selected stoichiometric molar ratio of citric acid to sodium carbonate is 4:3 or more (2× citric acid). The inflation curve evaluated for sodium carbonate mixture uses approximately 5× excess of citric acid with a stoichiometric balance of 10:3 (citric acid/sodium carbonate), where the selected portion of citric acid is about 5 times the concentration of citric acid in the balanced reaction. In the experiment, a 2-mil PEBAX balloon containing 81-mg effervescent mixture containing 5× stoichiometric excess citric acid was used. Among the mixture, 11.3 mg (0.107 mol) are sodium carbonate (106 g/mol) and 69.7 mg (0.36 mol) is citric acid (192 g/mol) which gives an acid to base molar ratio of about 10:3 (0.36/0.107).

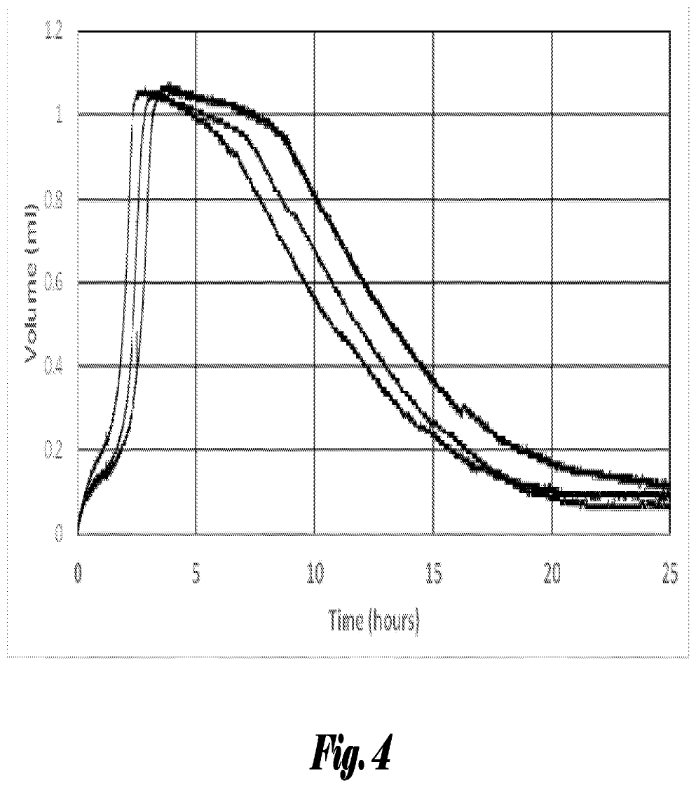

The inflation curves for six samples with the 5× stoichiometric ratio are shown in FIG. 4, where these inflation curves now illustrate the desirable mono-modal inflation characteristics. While 5× stoichiometric excess citric acid is used in the evaluation, any citric acid/sodium carbonate mixture with a 2× (4:3 molar stoichiometric ratio) or more excess of citric acid works satisfactorily.

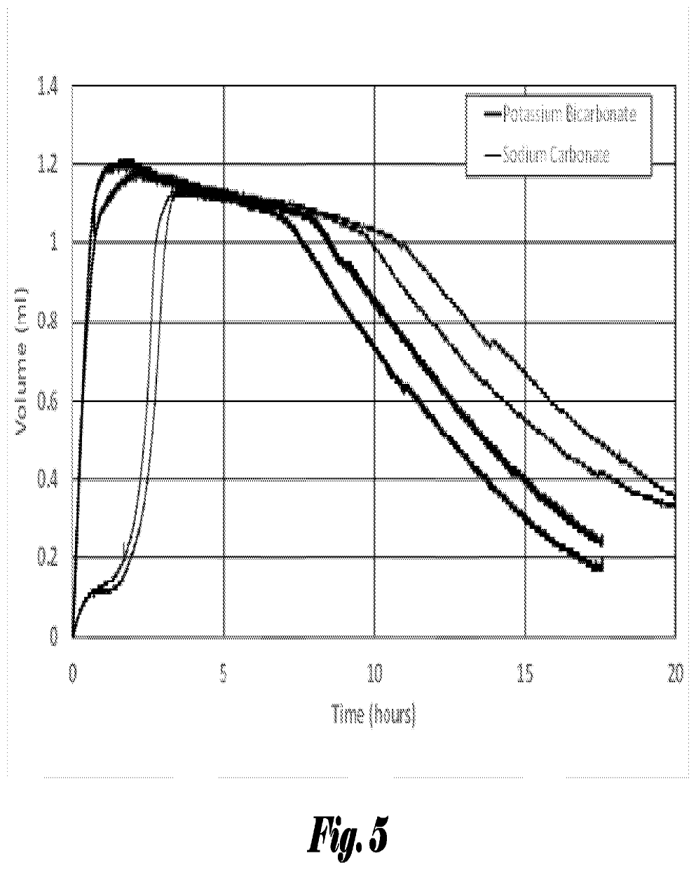

In yet another study, the balloon inflation curves are compared between potassium bicarbonate mixture and sodium carbonate mixture. In this comparison, a PEBAX tube balloon with 2-mil thickness was used. The tube was filled with 15 mg (0.14 mmol) sodium carbonate and 70 mg (0.36 mmol) citric acid (192 g/mol) or 10 mg (0.10 mmol) potassium bicarbonate (100 g/mol) and 30 mg (0.15 mmol) citric acid where both mixtures include an approximate 5× molar excess of citric acid relative to their balanced stoichiometric reactions (Equations (1) and (2)). The gas inflation curves for both systems are shown in FIG. 5. As shown in FIG. 5 for the sodium carbonate effervescent mixture, the inflation starts about 2 hours after the tube is exposed to the simulated stomach-intestine environment. Since the capsule needs to exit the strong acid stomach environment before it starts to inflate this 2 hours delay is the desired initiation time for balloon inflation. However, the potassium bicarbonate mixture starts to inflate much sooner and reaches full inflation around 1 hour. Therefore, the potassium bicarbonate mixture encounters an issue of early inflation if used without an enteric coated shell.

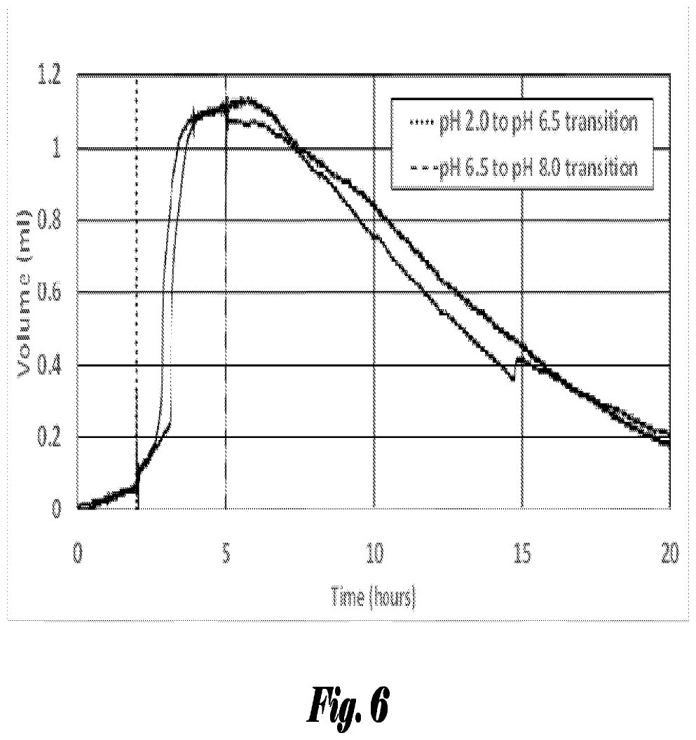

In order to delay the inflation initiation time for the potassium bicarbonate mixtures, the balloon is enclosed with an enteric or an enteric coated shell to delay the starting time of inflation. Addition of the enteric shell makes the balloon inflation not only time controlled but also pH dependent. For example, a full-shell can be used to enclose the balloon as well as the whole capsule device. In another example, the balloon can be attached to one end of the capsule device and a half-shell is capped on this end of the capsule device to enclose the balloon. As shown in FIG. 6, with a proper enteric or an enteric coated shell, the inflation initiation is delayed to a desired anatomical location (starting pH). Again, the tube is filled with 10 mg (0.1 mmol) potassium bicarbonate (100 g/mol) and the potassium bicarbonate mixture used in this experiment includes an excess of citric acid (30 mg, 0.15 mmol) with a citric acid/potassium bicarbonate molar ratio of 3:2 which is an about 4.5× times excess of the balanced stoichiometric ratio of 1:3 (Equation 2).

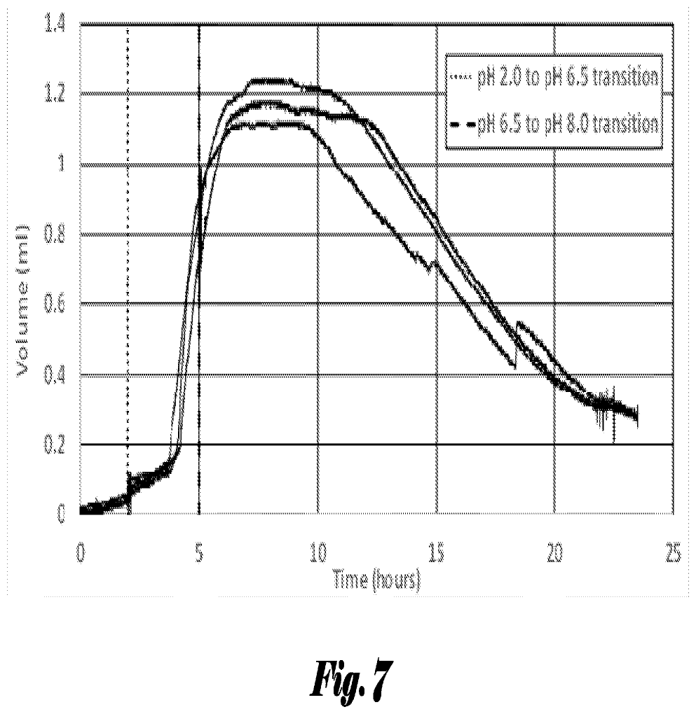

In addition, desiccants such as PEG (polyethylene glycols) can be used inside the balloon to the inflation starting time at a given pH. The PEG could be of any molecular weight, morphology, or structure (e.g. linear, star-shaped). FIG. 7 illustrates the balloon inflation curves for potassium bicarbonate mixture with PEG using an enteric or an enteric coated shell. In this experiment, the tube is filled with 10 mg (0.10 mmol) potassium bicarbonate and the effervescent potassium bicarbonate mixture used in this experiment includes stoichiometric excess of citric acid (30 mg, 0.15 mmol). The effervescent potassium bicarbonate mixture used in this experiment is mixed with 15 mg PEG (MW 10,000 Daltons, semi-crystalline, 4-arm structure). Compared to the corresponding balloon inflation/deflation curves for effervescent potassium bicarbonate mixtures with an enteric or an enteric coated shell but without PEG, the balloon inflation starting time for potassium bicarbonate mixture with PEG and an enteric or an enteric coated shell at a given pH is delayed further.

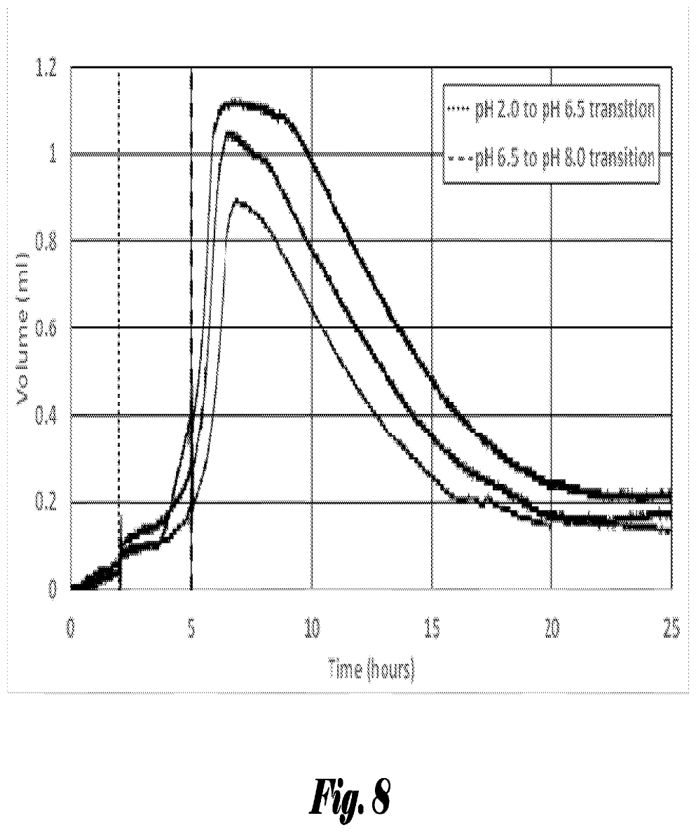

If further control (delay) of gas inflation starting time is desired for the sodium carbonate mixture, the tube with the sodium carbonate mixture (with excess citric acid) can also be enclosed in an enteric or an enteric coated shell. FIG. 8 illustrates the gas inflation curves for sodium carbonate mixture with an enteric or an enteric coated shell. Compared to the gas inflation curves for sodium carbonate mixture in FIG. 5, the gas inflation curves for sodium carbonate mixture with an enteric or an enteric coated shell in FIG. 8 is controlled by the pH and therefore have a delayed starting time. Addition of the enteric shell makes the balloon inflation not only time controlled but also pH dependent.

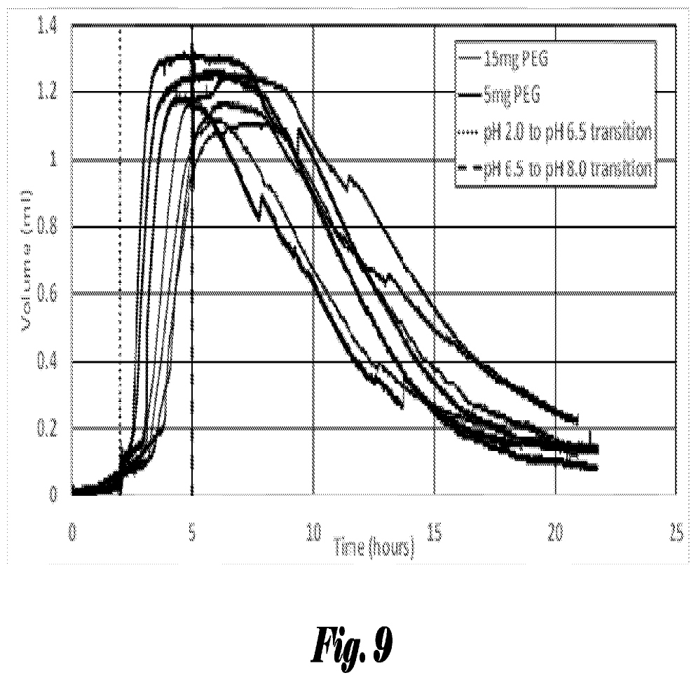

In another embodiment, a thin PEBAX balloon (1 mil thickness) is used to allow faster balloon deflation. The thin balloon can be used in combination with different amounts of PEG, or other balloon desiccants, in order to create a faster reaction that is still sufficiently delayed to inflate. In one experiment, the balloon inflation/deflation performance of effervescent mixtures using 10 mg (0.1 mmol) potassium bicarbonate mixed with citric acid and either 5 mg or 15 mg of PEG are compared as shown in FIG. 9. In this experiment, the balloon filled with the potassium bicarbonate effervescent mixture is also covered by an enteric or an enteric coated shell. As shown in FIG. 9, the inflation of these enteric protected balloons containing potassium bicarbonate mixtures (10 mg potassium bicarbonate with 30 mg citric acid) and either 5 mg or 15 mg PEG does not start for at least 2 h in pH 2. Further, at higher pH as shown in FIG. 9, the inflation starting time is further delayed for the balloon with a larger amount of PEG.

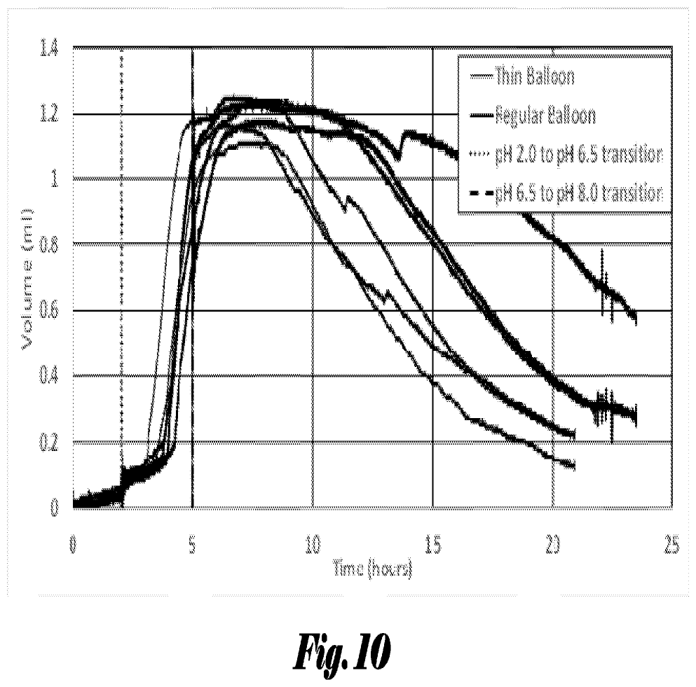

In yet another experiment, the gas inflation curves for thin balloon (i.e., 1 mil thickness) are compared with these for the regular balloon (i.e., 2 mil thickness) as shown in FIG. 10. In this comparison, potassium bicarbonate is used along with citric acid mixed with PEG. For the thin balloon, the balloon contains 55 mg (0.55 mmol) potassium bicarbonate with a stoichiometric excess of citric acid, and mixed with PEG (5 to 25 mg). For the regular balloon, the balloon contains 55 mg (0.55 mmol) potassium bicarbonate with stoichiometric excess of citric acid, and mixed with PEG (5 to 25 mg). As shown in FIG. 10, the gas inflation curves for the regular balloon have similar inflation characteristics as those of thin balloons. However, the regular thicker balloon has longer deflation times.

In the invention, both sodium carbonate and potassium bicarbonate are identified to provide a more thermally stable effervescent mixture than the conventional sodium bicarbonate comprising effervescent mixtures. Accordingly, sodium carbonate and potassium bicarbonate are disclosed as two candidate effervescent materials used to inflate a balloon, where the balloon is attached to a capsule device so as a means to control the specific gravity of a target capsule device.

While both sodium carbonate and potassium bicarbonate are more thermally stable, the balloon inflation curves often exhibit bimodal behavior, which is not ideal for specific gravity control of a target capsule device. In order to overcome this issue, excess citric acid is used in both the sodium carbonate and the potassium bicarbonate mixtures. Accordingly, the sodium carbonate mixture as well as the mixture with potassium bicarbonate and an about 5× stoichiometric molar excess of citric acid have been shown to exhibit consistent mono-model gas inflation curves. Nevertheless, sodium carbonate mixture and potassium bicarbonate mixture with a stoichiometric molar excess of acid relative to the balanced equations (1) and (2) equal to 2× or more will produce consistent mono-modal behavior.

While sodium carbonate mixture and potassium bicarbonate mixture with excess citric acid have the thermal stability characteristics and bi-modal gas inflation curves, these effervescent materials need to be tailored to provide the desired gas inflation curves as needed for controlling the specific gravity of a target capsule device. For potassium bicarbonate effervescent mixtures, the inflation initiation time is typically too soon for a capsule device intended for the lower gastrointestinal tract and balloons using potassium bicarbonates effervescent mixtures will start to generate CO₂ (g) before the capsule device exits the stomach. In order to delay the inflation initiation time, various means are disclosed. In one embodiment, enteric coating it used to delay the inflation starting time. For example, a full-shell can be used to enclose the balloon as well as the capsule device. In another example, the balloon can be attached to one end of the capsule device and a half-shell is capped on this end of the capsule device to enclose the balloon. In another embodiment, desiccants such as polyethylene glycols (PEG), starch, or other hydrophilic materials such silicates, magnesium sulfates, or Drierites can be used to delay the inflation starting time. For example, the potassium bicarbonate can be mixed with excess citric acid and PEG. Furthermore, the use of enteric coating and PEG can be combined. The inflation starting time means may also be applied to sodium carbonate.

In yet another embodiment, a thin balloon (1 mil thickness) is used to allow faster balloon deflation. The thin balloon can also be used in combination with different amounts of PEG and/or enteric coated shells in order to create a faster reaction that is still sufficiently delayed to inflate or targeted to the desired anatomical location through pH control.

In yet another embodiment, other acids than citric acid, ideally crystalline, anhydrous, low molecular weight and water soluble are used to allow thermally stable effervescent mixtures. Examples of other acids include but are not limited to tartaric acids and monocalciumphosphate (Ca(H₂PO₄)₂). The alternative acids can also be used in combination with different amounts of PEG, thin balloons, and enteric coatings in order to create a more controlled balloon inflation reaction that is still sufficiently delayed to inflate and thermally stable and/or targeted to the desired anatomical location through pH control. The stoichiometric reaction of monocalciumphosphate with potassium bicarbonate are outlined below:

14KHCO₃+5Ca(H₂PO₄)₂→14CO₂+Ca₅(PO₄)₃OH+7K₂HPO₄+13H₂O

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A capsule device with heat-stable specific gravity control, comprising:
a capsule unit adapted to be swallowable by a human subject; and
an inflatable balloon comprising a heat-stable effervescent formulation inside the inflatable balloon, wherein the heat-stable effervescent formulation is substantially free from thermal degradation up to a transportation temperature or shelf-life temperature belonging to a temperature range including 40° C., and wherein the inflatable balloon is attached to the capsule unit; and
wherein after the capsule unit with the inflatable balloon attached is swallowed, the inflatable balloon starts to inflate so as to lower specific gravity of a combination of the capsule unit and the inflatable balloon when the inflatable balloon is exposed to body fluid and the body fluid gets in touch with the heat-stable effervescent formulation inside the inflatable balloon; and
wherein the heat-stable effervescent formulation comprises a base with an excess acid.

2. The capsule device of claim 1, wherein the base comprises sodium carbonate, potassium bicarbonate or both.

3. The capsule device of claim 2, wherein the excess acid selected is crystalline or semi-crystalline, anhydrous, low molecular weight, and water soluble.

4. The capsule device of claim 3, wherein the excess acid belongs to a group comprising citric acid, tartaric acids and monocalciumphosphate (Ca(H₂PO₄)₂).

5. The capsule device of claim 2, wherein any excess acid relative to the base corresponds to at least two times a balanced stoichiometric molar ratio.

6. The capsule device of claim 2, wherein when the sodium carbonate is used, the sodium carbonate is contained in the inflatable balloon with the excess acid relative to a base of at least about five times a balanced stoichiometric molar ratio.

7. The capsule device of claim 2, wherein when 10-25 mg potassium bicarbonate is used, the 10-25 mg potassium bicarbonate is contained in the inflatable balloon with an excess acid relative to a base of at least about five times a balanced stoichiometric molar ratio.

8. The capsule device of claim 2, wherein the inflatable balloon is enclosed in an enteric or enteric coated shell to control inflation starting time.

9. The capsule device of claim 8, wherein when the potassium bicarbonate is used, the excess acid selected is crystalline or semi-crystalline, anhydrous, low molecular weight, and water soluble.

10. The capsule device of claim 9, wherein the excess acid belongs to a group comprising citric acid, tartaric acids and monocalciumphosphate (Ca(H₂PO₄)₂) mixed with polyethylene glycols (PEG) or an alternative desiccant to further control the inflation starting time.

11. The capsule device of claim 10, wherein about 10-25 mg potassium bicarbonate along with the excess acid relative to a base of about three six times a balanced stoichiometric molar ratio and mixed with about 5-25 mg PEG or alternative desiccant are contained in the inflatable balloon.

12. The capsule device of claim 8, wherein the enteric or enteric coated shell corresponds to a half shell to enclose the inflatable balloon between the half shell and a portion of the capsule unit.

13. The capsule device of claim 8, wherein the enteric or enteric coated shell corresponds to a full shell to enclose the inflatable balloon and an entire capsule unit.

14. The capsule device of claim 2, wherein when the potassium bicarbonate is used, the potassium bicarbonate along with the excess acid is mixed with polyethylene glycols (PEG) to further control the inflation starting time.

15. The capsule device of claim 2, wherein the inflatable balloon comprises a thickness of about 0.5-5 mil.

16. The capsule device of claim 2, wherein the inflatable balloon comprises a thickness about 0.5-2 mil.

17. The capsule device of claim 2, wherein the capsule unit comprises a camera to capture images while the capsule unit travels in a gastrointestinal tract of the human subject.

18. The capsule device of claim 1, wherein the capsule device is stored and/or transported through including a temperature range from 40° C. to 60° C.

19. The capsule device of claim 18, wherein the capsule device is stored and/or transported through includes a temperature range from 40° C. to 50° C.

20. The capsule device of claim 1, wherein the temperature range corresponds to 40° C. to 60° C.

21. A heat-stable effervescent formulation, comprising:
a target effervescent with an excess acid, wherein the excess acid selected is crystalline/semi-crystalline, anhydrous, low molecular weight, and water soluble; and
wherein the heat-stable effervescent formulation is substantially free from thermal degradation in an environment below a transportation temperature or shelf-life temperature belonging to a temperature range including 40° C.; and
wherein the heat-stable effervescent formulation comprises a base with an excess acid.

22. The heat-stable effervescent formulation of claim 21, wherein the target effervescent comprises sodium carbonate, and/or potassium bicarbonate.

23. The heat-stable effervescent formulation of claim 21, wherein any excess acid relative to a base corresponds to at least two times a balanced stoichiometric molar ratio.

24. The heat-stable effervescent formulation of claim 21, wherein the excess acid belongs to a group comprising citric acid, tartaric acids and monocalciumphosphate (Ca(H₂PO₄)₂).
